(19) United States
(12) Patent Application Publication
(10) Pub. No.: US 2011/0153005 A1
(43) Pub. Date: Jun. 23, 2011

(54) MEDICAL IMPLANT, COATING METHOD AND IMPLANTATION METHOD

(76) Inventor: Claus Harder, Uttenreuth (DE)

(21) Appl. No.: 12/972,102

(22) Filed: Dec. 17, 2010

Related U.S. Application Data
(60) Provisional application No. 61/288,346, filed on Dec. 21, 2009.

(51) Int. Cl. A61F 2/82

(52) U.S. Cl. 623/1.46

(57) ABSTRACT

One example of the invention relates to a medical implant having a base body comprising a first end and a second end, which are arranged at opposite ends of the base body in a main direction of the extent of the base body, and a coating, such that the coating has at least one active substance with an active substance gradient. It is proposed that the quantity of active substance shall decrease from the first end to the second end, at least in the main direction of extent of the base body.

### CROSS REFERENCE

The present application claims priority on U.S. Provisional Application No. 61/288,346 filed on Dec. 21, 2009; which application is incorporated by reference herein.

### TECHNICAL FIELD

An example of the invention relates to a medical implant and a method, in particular a coating method for coating a medical implant, and a method for implantation of a medical implant in an animal or human body.

### BACKGROUND

Implants are used in medicine to be introduced into an animal or human body permanently or at least for a prolonged period of time to fulfill replacement functions. Examples include cardiac pacemakers, cerebral pacemakers for Parkinson's patients, cardiac implants, cochlear implants, retinal implants, dental implants, joint replacement implants, vascular prostheses and stents. Stents coated with an active substance, in particular, so-called drug-eluting stents (DES), have recently been used with increasing frequency in the field of cardiovascular disease to reduce the sequelae of procedures, such as a restenosis or reocclusion of blood vessels, through local administration of an active substance following dilatation of stenosed coronary vessels by means of angioplasty and stabilization by stent implantation.

It is known from DE 10 1006 038239 A1 that a stent may have a so-called layer-by-layer coating with a radial active substance gradient, so that one or more active substances are eluted in various time-dependent doses and/or in one or more different time slots. Furthermore, this prior art document discloses a method for producing the coated stent, in which the active substance gradient is produced by applying different active substance/polymer mixtures layer by layer radially. US 2005 0075714 A1 also discloses such a stent as well as a comparable method.

US 2008 0195079 A1 discloses a stent having three areas of different coatings, such that the areas at the ends of the stent have the same drug concentration, which is higher than the drug concentration in the area between the two ends.

### SUMMARY

A feature of some invention embodiments is to create a medical implant and a method for implantation of a medical implant in an animal or human body that will permit enhanced administration of an active substance and will achieve a targeted mechanism of action. Furthermore, some invention embodiments provide a method for coating a medical implant

This feature is achieved by the elements of the independent claims. Example embodiments of the invention are derived from the additional claims, the drawings, and the description.

An example of the invention is directed to a medical implant having a base body with a first end and a second end, arranged opposite one another in the main direction of the extent of the base body (by way of example, the distal opposing ends of the implant), and a coating, such that the coating has at least one active substance with an active substance gradient. Other example embodiments include methods for making an implant, and methods for implanting one.

### DESCRIPTION OF THE DRAWINGS

The invention is described in greater detail below on the basis of exemplary embodiments illustrated in the drawings, in which schematically:

FIG. 1 shows an inventive medical implant in a view from above with a coating,

FIG. 2 shows a schematic diagram of a section through the medical implant from FIG. 1 with a coating in a first embodiment,

FIG. 3 shows a diagram of an active substance gradient,

FIG. 4 shows the implant from FIG. 1 implanted in a cavity,

FIG. 5 a-c show three simulations of elution of an active substance of three medical implants with different coatings according to the state of the art,

FIG. 6 shows a schematic diagram of a coating method of the medical implant from FIG. 1, and

FIG. 7 shows a schematic diagram of a section through a medical implant with an alternative embodiment of the coating.

Elements that are functionally the same or have the same effect are each labeled with the same reference numerals in the figures. The figures show schematic diagrams of the invention. They illustrate nonspecific parameters of the invention. In addition, the figures show only typical embodiments of the invention are not intended to restrict the invention to the embodiments shown here.

### DETAILED DESCRIPTION

The discovery that the quantity of active substance deposited at a certain location on the vascular wall not only depends on the local elution of the active substance at this location but also is influenced by the medium flowing through the stent because this medium conveys the active substance, which enters the stream there, downstream proximally away from this location and can deposit it at this location. In particular it has been discovered that when using active substances having a narrow therapeutic window, this effect plays a role that must be taken into account in achieving targeted deposition of the active substance.

Some embodiments of the invention direct that the quantity of active substance should decrease from the first end to the second end, at least in the main direction of extent of the base body. In this context, the term “decrease” should be understood to refer to a continuous or quasi-continuous reduction in the amount of active substance, which declines by stages, monotonically and/or strictly monotonically. The phrase “end of the base body” should be understood here to refer to an area of the implant, to which is connected an implant structure and/or a base body structure, such as a wire mesh, in only one direction. The phrase “main extent” here should be understood to refer to the length of the base body and/or, in the implanted state of the implant, a direction along the direction of flow of a fluid medium. The phrase “opposite ends” should be understood here to refer in particular to ends situated at different ends relative to the direction of flow, i.e., at the beginning and end (e.g., upstream and downstream portions) of the implant. In the implanted state of the implant in a cavity body through which a fluid medium is flowing in an animal or human body, the quantity of active substance in the coating may thus either increase or decrease in the axial direction of flow, depending on the direction of implantation. An embodiment of the invention makes available a medical implant, which is coordinated with the parameters of the implantation site, such as the absorption rate of active substance by a cavity wall adjacent to or in contact with the implant and/or the flow rate of a medium flowing through the cavity and/or the implant. In this way, a desired homogeneous concentration of active substance is achieved in the surrounding cavity wall, so that local overdosing or underdosing can be prevented in an especially advantageous manner.

Furthermore, an “implant” should be understood to be a body which fulfills a replacement function when implanted in an animal or human body either permanently or for an extended period of time. All medical implants that seem expedient to those skilled in the art would be conceivable here, e.g., a cardiac pacemaker, a cerebral pacemaker, a cardiac implant, a cochlear implant, a retinal implant, a dental implant, a joint prosthesis implant, a vascular prosthesis or, especially advantageously, an embodiment of the medical implant as a stent, in particular as a coronary stent is proposed as being especially advantageous. The implant and/or the stent preferably comprises a depot for a pharmaceutically active substance, so that dosing of the active substance may advantageously be coordinated with the needs of the vascular wall surrounding the implant, taking into account the rate of blood flow.

Furthermore, in this context, the term “base body” should be understood to refer to a structure such as a wire mesh (although other structures are possible in addition to a wire mesh), which essentially forms the shape and/or form of the implant and/or stent. Furthermore, the base body is preferably manufactured from a metallic material or from a combination of several metallic and non-metallic materials such as iron, magnesium, nickel, tungsten, titanium, zirconium, niobium, tantalum, zinc, silicon, lithium, sodium, potassium, calcium, manganese or any other material that seems suitable to those skilled in the art. A zinc-calcium alloy or a shape memory material, such as a nickel-titanium alloy or a copper-zinc-aluminum alloy, preferably Nitinol, are also possible in invention embodiments. In addition, it may be advantageous for at least some applications if the base body comprises at least cobalt and/or chromium, preferably in the form of stainless steel and/or a Cr—Ni—Fe steel (preferably the alloy 316L) or a Co—Cr steel. Through this embodiment, it is possible to provide an implant that leads to satisfactory coating results and has good dilatability and an advantageous flexibility combined with a high stability.

In other embodiments the base body of the implant consists at least partially of a plastic, a ceramic, or a biodegradable material.

Furthermore, in this context a “coating” should be understood to refer to at least partial sheathing or covering of the implant, preferably a stent and/or filling and/or loading comprising at least one recess, preferably in the form of a hole, a cavity or a basket in or on the implant, with a suitable matrix comprising at least one active substance. Preferred polymers for a polymer matrix of embodiments of the implant are selected, depending on need, from the following groups:
nonresorbable/permanent polymers, such as:
polypropylene, polyethylene, polyvinyl chloride, polyacrylates (polyethyl and polymethyl acrylates, polymethyl methacrylate, polymethyl-co-ethyl acrylate, ethylene/ethyl acrylate), polytetrafluoroethylene (ethylene/chlorotrifluoroethylene copolymer, ethylene/tetrafluoro-ethylene copolymer), polyamides (polyamidimide, PA-11, -12, -46, -66), polyetherimide, polyethersulfone, poly(iso)butylene, polyvinyl chloride, polyvinyl fluoride, polyvinyl alcohol, polyurethane, polybutylene terephthalate, silicones, polyphosphazene, polymer foams (e.g. from carbonates, styrenes), as well as the copolymers and blends of the selected classes and/or the class of thermoplastics and elastomers in general;
resorbable/bioresorbable/degradable polymers, such as: polydioxanone, polyglycolide, polycaprolactone, polylactides [poly-L-lactide, poly-D,L-lactide, and copolymers as well as blends, such as poly(L-lactide-co-glycolide), poly(D,L-lactide-co-glycolide), poly(L-lactide-co-D,L-lactide), poly(L-lactide-co-trimethylenecarbonate)], triblock copolymers, polysaccharides [chitosan, levan, hyaluronic acid, heparin, dextran, cellulose, etc.], polyhydroxyvalerate, ethylvinyl acetate, polyethylene oxide, polyphosphorylcholine, fibrin, albumin, polyhydroxybutyric acid (atactic, isotactic, syndiotactic and blends thereof), etc.

The coating preferably contains at least one polylactide, such as poly-L-lactide, so that a biocompatible and especially low-weight coating can be provided.

An “active substance” should be understood here to be a substance which induces and/or suppresses a biochemical and/or biological response in the animal or human body. In this sense, “an active substance” may also be used as synonymous with a “drug” and/or a “pharmaceutical agent.” It is preferred that at least one active substance is selected from the group consisting of lipid regulators (fibrates), immunosuppressants, immunomodulators, vasodilators (sartans), calcium channel blockers, calcineurin inhibitors (tacrolimus), antiphlogistics (glucocorticoids, cortisone, diclofenac), anti-inflammatories (imidazoles), anti-allergies, oligonucleotides (dODN), estrogens (genistein), endothelializing agents (fibrin), steroids, proteins/peptides, proliferation inhibitors, analgesics, antirheumatics, cytostatics. A medical implant and/or a stent having a broad spectrum of applications can be made available in this way.

In another embodiment, the invention has proved to be advantageous in use of an active substance having a narrow therapeutic window. The phrase “narrow therapeutic window” as used here should be understood to refer to a narrow therapeutic scope, where “therapeutic scope” refers to the ratio between a therapeutic dose and a toxic dose of the active substance. For example, through the inventive coating profile of the medical implant, it is possible to advantageously minimize the risks of substances having a narrow therapeutic window by utilizing critical limits with respect to overdosing or underdosing. An especially advantageous result can be achieved if at least one active substance is the immunosuppressant sirolimus.

In addition, one embodiment proposes that the active substance gradient should be adjusted through the thickness of the coating. “Thickness” here defines a radial measurement of the coating with respect to a midpoint of the implant, the midpoint being a line along the longitudinal axis of the implant, generally positioned in the direction of fluid flow. An “active substance gradient” should be understood here to refer to a change in the quantity of active substance between two points and, particularly, between the first end and the second end. The thickness of the coating, and thus also the quantity of active substance, at the first end is preferably greater than the thickness of the coating at the second end of the base body. Due to the variable thickness, preferably decreasing monotonically along the length in the main direction of extent of the base body, the active substance gradient can be established and adjusted through design techniques.

The active substance gradient is preferably set by means of one or more active substance concentrations along the coating. The radial thickness of the coating along the main direction of extent of the implant is the same over the entire length but the quantities of active substance differ at the first end and at the second end. Through the implementation of multiple active substance concentrations, the implant may advantageously be designed and produced to be homogeneous in its dimensions.

Furthermore, it is proposed that in the implanted state in a cavity, the first end should be positioned at the upstream end in an axial direction of flow. In this context, the term “cavity” should be understood in particular to refer to a cavity in an animal or human body such as a tubular structure and/or a tubular vessel through which a fluid medium and/or a body fluid such as lymph, bile, urine, lacrimal fluid, saliva, cerebrospinal fluid, or blood flows. An “upstream end” should be understood here to refer in particular to a proximal end and/or an end which comes in contact with the fluid medium at an earlier point in time than another end and/or the second end and/or a distal end. Put another way, fluid flows from an upstream end towards the downstream end. Also, the term “end” when used in this context is not intended to refer to the absolute start or finish of a cavity—any section of a cavity may be considered to have an upstream and downstream end. In the implanted state, the implant is preferably situated with an inside surface of the wall of the cavity and/or a vascular wall at least partially for support thereof. Put another way, in an implanted state in use within a cavity an implant of the invention may be in contact with the surrounding cavity—an exterior surface of the implant may lie adjacent and in supporting contact with the cavity wall. Through this orientation, the implant is thus designed to be optimized in particular with regard to both flow and the varied coating profile.

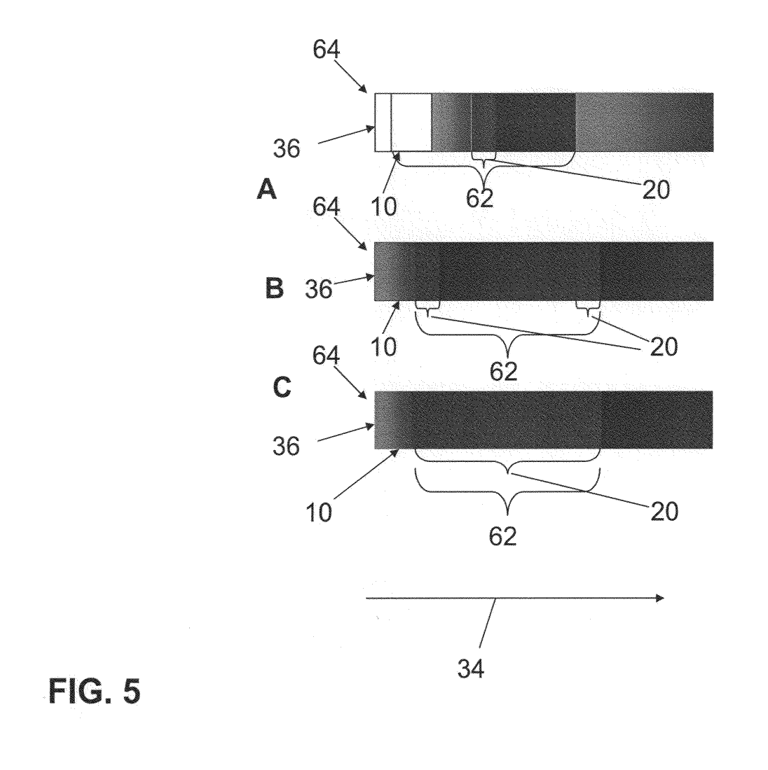

In an example medical implant embodiment for elution of at least one active substance in a human and/or animal body, such that the at least one active substance is elutable so that a desired local concentration distribution is achieved in an adjacent cavity wall, then this makes available an implant that is especially valuable therapeutically and has an especially balanced spectrum of effects and/or potential. The phrase “desired local concentration distribution” should be understood to mean that a concentration distribution or an active substance distribution is coordinated with at least one parameter of the implant-cavity system. The parameter may refer to the active substance, the cavity, the cavity wall or the medium and/or flow medium surrounding and/or flowing through the cavity and may represent a type of active substance, a volume, an absorbability, an elasticity, a flow rate, etc. The active substance distribution may be heterogeneous or homogeneous. The desired local concentration distribution of a respective active substance distribution in the coating may be ascertained and/or optimized by simulation (see also FIG. 5).

As used herein the term “elution” is intended to broadly refer to release. Elution may occur in short, long or intermediate time periods depending on a desired application as well as other factors. In at least some embodiments, elution of the active substance occurs during a time period of several minutes, in others an hour or more, and in others longer periods of time. Various methods of elution or release of the active substance from the coating or its polymer matrix are differentiated according to the design of the inventive implant comprising the implant base body, the carrier matrix and the active substance:
Active substances may be applied directly to the implant base body, e.g., to a metallic stent base body, either by covalent bonding or by ionic or van der Waals' interaction.
Covalent bonding of the active substance(s) to a coating is also contemplated. The active substance(s) is/are then released via hydrolytic or enzymatic cleavage of the bond.
Active substances may be incorporated into cavities in the stent material and provided with a polymeric cover layer. Elution takes place either by diffusion processes though the cover layer or directly, in the case of a layer that dissolves immediately after implantation.
The active substance may be contained within a porous, usually inorganic, carrier matrix and released by it into the tissue.
An active substance may be released by swelling of its carrier matrix.
Elution can be achieved by erosion of a carrier matrix comprising biodegradable polymers.
The active substance may be applied on top of a first layer and underneath a later applied second layer. The active substance thus embedded between two polymer layers may elute out of this sandwich structure through diffusion processes.
A blend of polymer and active substance may be applied to the implant base body, particularly in the case of a stent, thus enabling its release through diffusion.
A topcoat, i.e., an additional polymer layer without an active substance, may be situated around a polymer layer in which the active substance is incorporated, leading to retardation of the elution process until after the overlaid polymer topcoat layer has dissolved.

Furthermore, another invention embodiment is directed to a method, in particular, a coating method for coating a medical implant comprising a base body having a first end and a second end and the coating, which contains at least one active substance with an active substance gradient.

In one embodiment, a coating stream with a polymer/active substance mixture is moved at a variable rate from the first end to the second end. In this context, a “polymer/active substance mixture” should be understood to refer to any combination of one or more of the polymers already listed and one or more of the active substances already listed, or any other combination of similar materials relevant in the art. A “variable rate” should be understood here to refer to a changing rate, with one example being a linear or modified linear rate. For example, where the implant is a stent or preferably a coronary stent, because of the variable rate, different coating thicknesses are applied along a main direction of extent from the first end to the second end, or any other length in the direction of fluid flow, of the implant, resulting in different quantities of active substance at different axial positions of the implant and/or stent. The coating stream may be produced by any coating unit that seems appropriate to those skilled in the art, e.g., equipped with a two-substance nozzle (with or without forced conveyance), a single-substance nozzle, a rebound nozzle, a dosing valve, a rotary atomizer or an ultrasonic atomizer. In general, movement of the implant relative to the coating stream would also be conceivable. Furthermore, any other method of coating the implant expedient to those skilled in the art, e.g., by means of a roller application or by means of an immersion reservoir, is also contemplated. Varying the concentration of active substance in the polymer/active substance mixture through different mixing conditions or other steps is also contemplated. In such embodiments the coating layer containing the polymer active substance is applied in a substantially even thickness over the implant between first and second ends, but the concentration gradient of the one or more active substances in the coating varies over the length. Through an example embodiment, the active substance gradient can be adjusted and/or established easily through the design. Furthermore, the coating can be applied reliably and in a reliable process.

In an alternative embodiment, it is possible to provide for the coating stream to move from the first end to the second end at an increasing rate. In this way, the radial thickness of the coating becomes thinner along the length, starting from one coating thickness at the first end (where the stream is applied moving slowest to result in a high thickness), the proximal end in the implanted state of the coated implant, to the second end, the distal end (where the stream is applied moving fastest to result in a low thickness), so the quantity of active substance preferably declines monotonically, i.e., becomes smaller from the first end to the second end. A coating with a monotonically decreasing active substance gradient can be applied quickly and reliably by increasing the rate of application.

According to a preferred further embodiment, the coating stream is moved in the axial direction or in the circumferential direction relative to the medical implant, or both. In this embodiment, the coating stream is moved by a length Δz axially starting from the first end in the direction of the second end after a revolution of the implant around the circumference of the implant, i.e., by 360° around the implant's axis, where Δz is limited only by the design specifications of the coating device. By implementation of the movement in the axial direction and/or in the circumferential direction, the coating of the implant may be applied so that it is advantageously uniform and homogeneous.

Furthermore, an embodiment of the invention is directed to a method, in particular, an implantation method for implanting a medical implant in an animal or human body, with a first end and second end, such that the first end has a greater loading of active substance than the second end.

In one such embodiment, it is proposed that the first end should be arranged in a cavity at the upstream end in the axial direction of flow. The term “active substance loading” here should be understood to refer to the quantity of active substance per unit of area. In one embodiment the implant is a stent and preferably a coronary stent. The cavity is formed by a vessel, in particular a blood vessel and especially preferably a coronary artery. The active substance is any substance that appears suitable to those skilled in the art and in particular may be one or more of the substances mentioned in the introduction. Through the inventive embodiment, it is possible to make available an especially well-developed form of treatment, which is coordinated in particular with the in vivo requirements of an implantation site.

To further illustrate various aspects of invention embodiments, the figures will now be referenced. To avoid unnecessary repetition, reference is made to the respective description of the elements in the figures indicated above in the case of elements that are not described more specifically in a figure.

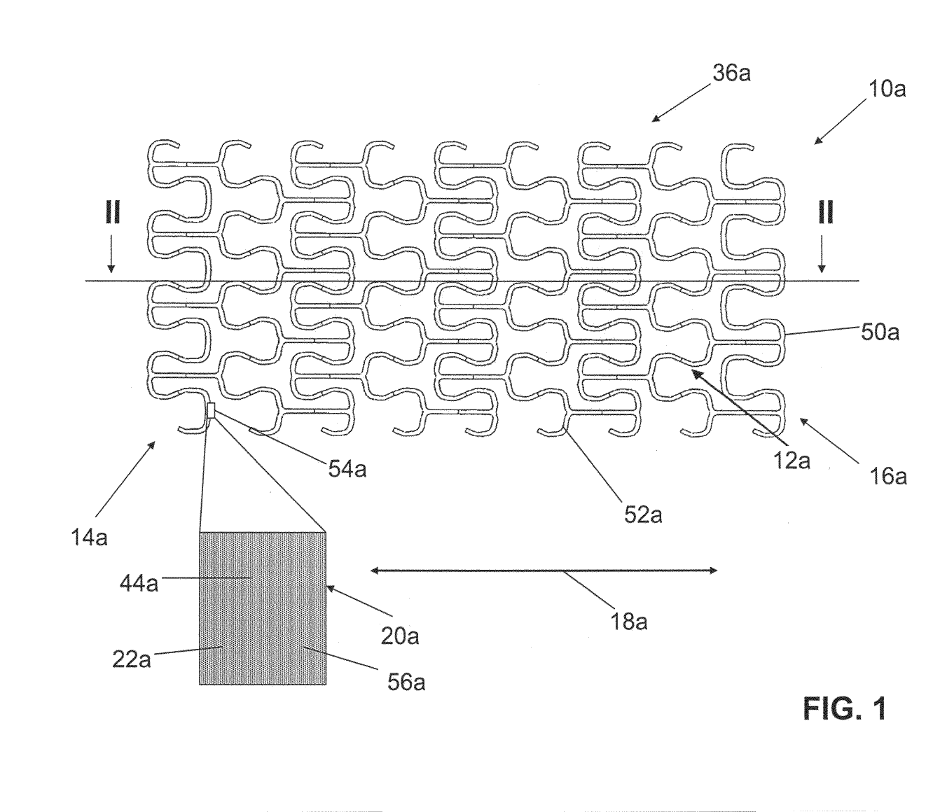

FIG. 1 shows in a view from above an example medical implant 10a in the form of a stent 36a, i.e., a coronary stent having a base body 12a made of chromium-cobalt steel, having as the base body structure a wire mesh 50a embodied by stent struts 52a. Furthermore, the base body 12a has a first end 14a and a second end 16a, which are arranged on opposite ends of the base body 12a in a main direction of extent 18a of the base body 12a. In general, the embodiment of the wire mesh 50a may be of any desired form that seems appropriate to those skilled in the art.

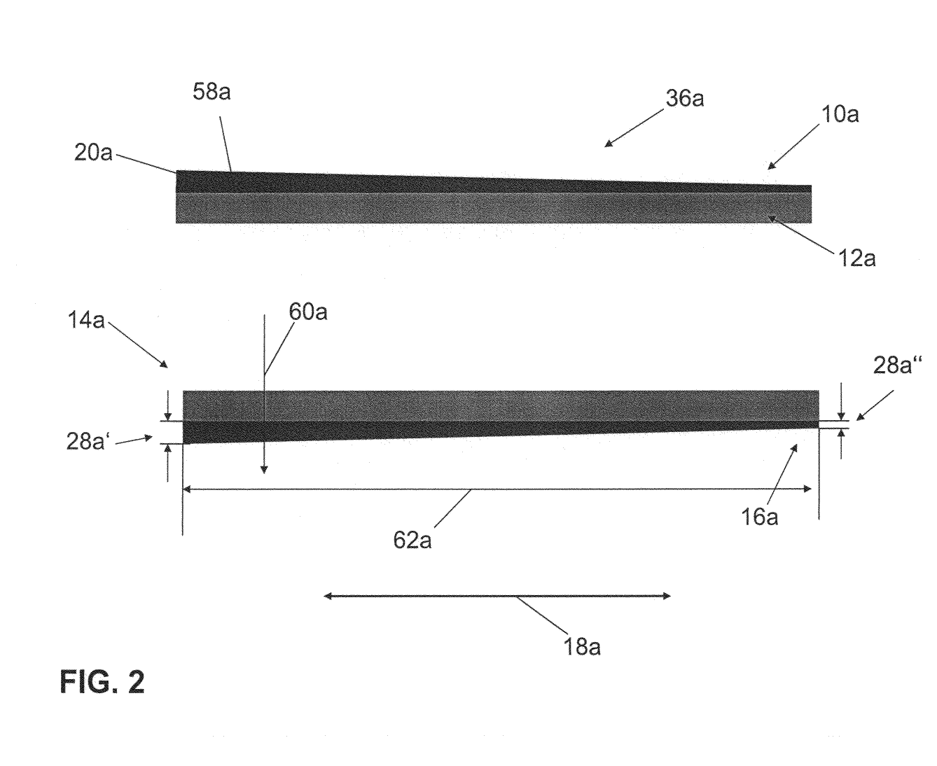

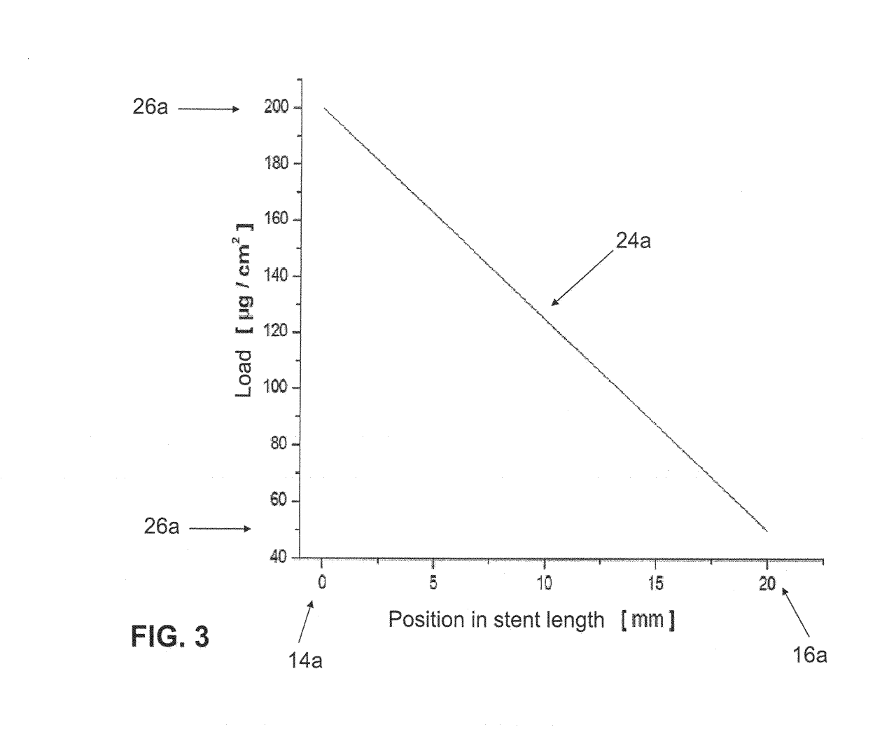

The implant 10a and/or the stent 36a is also coated with a coating 20a, which is introduced into cavities 54a of the stent struts 52a. These cavities 54a are distributed over the entire area of the stent struts 52a in a manner with which those skilled in the art are familiar, but only one cavity 54a is indicated here to yield a simpler diagram and is shown symbolically enlarged. The coating 20a is formed by a polymer/active substance mixture 44a, which contains the immunosuppressant sirolimus as the active substance 22a, and poly-L-lactide (PLLA) as the polymer matrix 56a. The active substance 22a thus has a narrow therapeutic window. Furthermore, the active substance 22a of the coating 20a has an active substance gradient 24a (FIGS. 2 and 3).

In FIG. 2 the medical implant 10a is shown schematically in a sectional diagram along line II-II in FIG. 1. The coating 20a is represented symbolically here to illustrate the coating profile on an exterior surface 58a of the base body 12a. The entire exterior surface of the implant base body may thus be coated in some embodiments, while in other embodiments only portions of the exterior surface are coated. The inside surfaces and side surfaces of the stent struts may also be coated, but for the sake of brevity, this is not shown in the drawing. In some embodiments these surfaces are not coated. The coating 20a has thicknesses 28a′, 28a″ which vary in a radial direction 60a along the main direction of extent 18a, resulting in an active substance gradient 24a through the thickness 28a′, 28a″ of the coating 20a. On the first end 14a of the base body 12a, the thickness 28a′ of the coating 20a is thicker than the thickness 28a″ on the second end 16a. Since the polymer/active substance matrix of the coating 20a has the same active substance concentration over the entire length 62a of the base body 12a, the quantity of active substance 26a in the main direction of extent 18a of the base body 12a declines from the first end 14a to the second end 16a and has a strictly monotonic decline (FIG. 3).

FIG. 3 shows a diagram of the active substance gradient 24a, where the position of the stent length in mm is plotted on the x axis and the load in μg active substance per cm² stent area is plotted on the y axis. It can be seen clearly here that the coating at the beginning of the implant 10a and/or at the first end 14a at the position at 1 mm has a coating of approximately 200 μg/cm² and at one end of the implant 10a and/or at the second end 16a at 20 mm has a coating of approximately 50 μg/cm². The quantities of active substance 26a decrease and thus the active substance 24a also decreases from the first end 14a to the second end 16a according to a strictly monotonic decline. In this example, the loading has decreased by a factor of 4 from first to second end (200 to 50 μg/cm²). It has been discovered that a loading difference of at a factor of 4 from first end (highest) to second end (lowest) is useful in many applications. Other invention embodiments, however, have loading differences that are less than 4, with one example being a difference of 2.

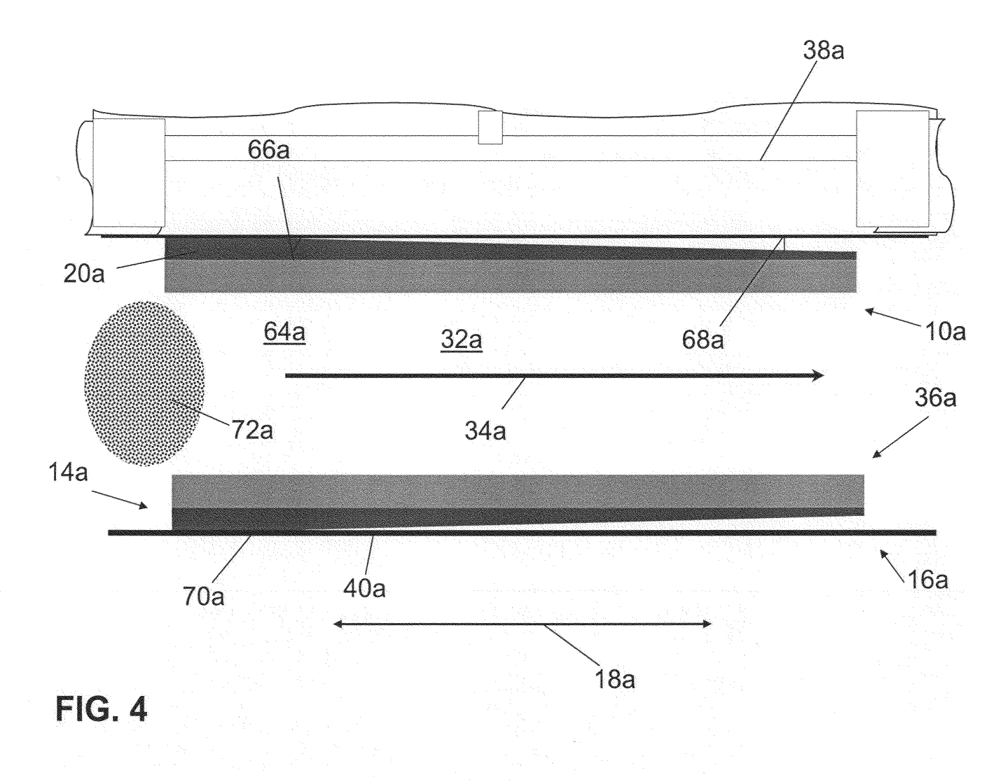

FIG. 4 shows the medical implant 10a, or the stent 36a, in an implanted state in a cavity 32a of a human body 38a. The implant 10a is introduced into the human body 38a by means of a method for implantation of the medical implant 10a and/or an implantation method in the human body 38a. The cavity 32a is formed by a tubular vessel, or a coronary artery 64a. At its outer lateral surface 66a, the implant 10a is in contact with an inside surface 68a of the cavity wall 40a, or vascular wall 70a of the coronary artery 64a (not represented correctly here by the symbolic and enlarged diagram of the coating 20a). The first end 14a, which has a higher active substance load than the second end 16a, is introduced into the cavity 32a, or the coronary artery 64a, so that at the upstream end, it flows through the cavity 32a in an axial direction of flow 34a of a fluid medium 72a and comprises a bodily fluid and/or blood

The medical implant 10a has an embodiment for eluting the active substance 22a in the human body 38a, such that the active substance 22a is eluted to yield a desired local concentration distribution in the adjacent cavity wall 40a. This desired local concentration distribution of the respective active substance distribution in the coating 20a can be determined and optimized by simulation.

FIG. 5 shows three simulations of elution of an active substance from three medical implants coated variously according to the prior art (http://medtechinsider.com/?p=4000) as an example. Such a simulation would also be conceivable for the stent 36a coated according to the present invention. The elution is described as Example C. A medical implant 10, or a stent 36, is implanted in a coronary artery 64, and a bodily fluid (not shown) flows through it in the direction of flow 34. Furthermore, the stent 36 is provided with a coating 20 over its entire length 62. The quantity of active substance deposited in the vascular wall is illustrated by means of gray shading. It can be seen that upstream from the stent 36 in the direction of flow 34, there is a small quantity of active substance due to elution and diffusion. In the area of the stent 36, elution of the quantity of active substance increases over its entire length 62, then decreasing downstream from the stent 36. Through such a simulation, it is possible to coordinate the coating with the parameters of the implant-cavity system.

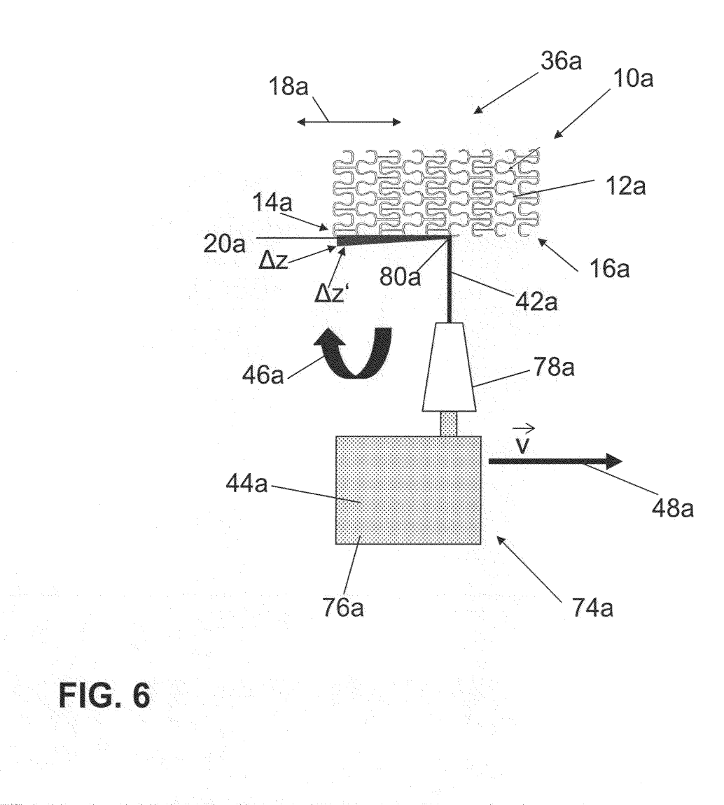

FIG. 6 shows schematically a device 74a for a method for coating a medical implant 10a in the form of a stent 36a with a base body 12a, which has a first end 14a and a second end 16a. Furthermore, the base body 12a has a coating 20a, which has an active substance 22a with an active substance gradient 24a. The device 74a has a reservoir 76a with the polymer/active substance mixture 44a, which is applied as a coating stream 42a to the implant 10a, or the stent 36a, by means of a nozzle 78a. In doing so, the coating stream 42a of the polymer/active substance mixture 44a is moved at a variable rate, preferably an increasing velocity, from the first end 14a to the second end 16a of the base body 12a. Due to the increase in velocity, the coating 20a becomes progressively thinner from a first position proximate to or at the first end 14a, to a second position 80a proximate to or at the second end 16a (coating result on the second end 16a not shown). Velocity can be varied in one or both of fluid flow through the nozzle, or movement of the nozzle relative to the implant. That is, if the nozzle moves at a constant velocity relative to the implant but coating material flows through the nozzle at a varying rate, a varying amount of coating material will be deposited on the implant along its length. If, on the other hand, coating material flows through the nozzle at a constant rate but the velocity of the nozzle is varied relative to the implant, a varying amount of coating material will likewise be deposited on the implant. In one embodiment, to achieve a complete coating of the implant 10a, the coating stream 42a is moved in the circumferential direction 46a and in the axial direction 48a by means of the device 74a relative to the medical implant 10a. A section Δz is coated only in the circumferential direction 46a around the entire circumference of the implant 10a, and then the coating stream 40a is moved further in the direction of the second end 16a by the distance Δz in the axial direction 48a to coat the next section Δz′ in the circumferential direction 46a. This is continued until the medical implant 10a has been coated completely. In other embodiments the implant 10a is moved relative to the nozzle, in one or both of the Δz direction and in the circumferential direction.

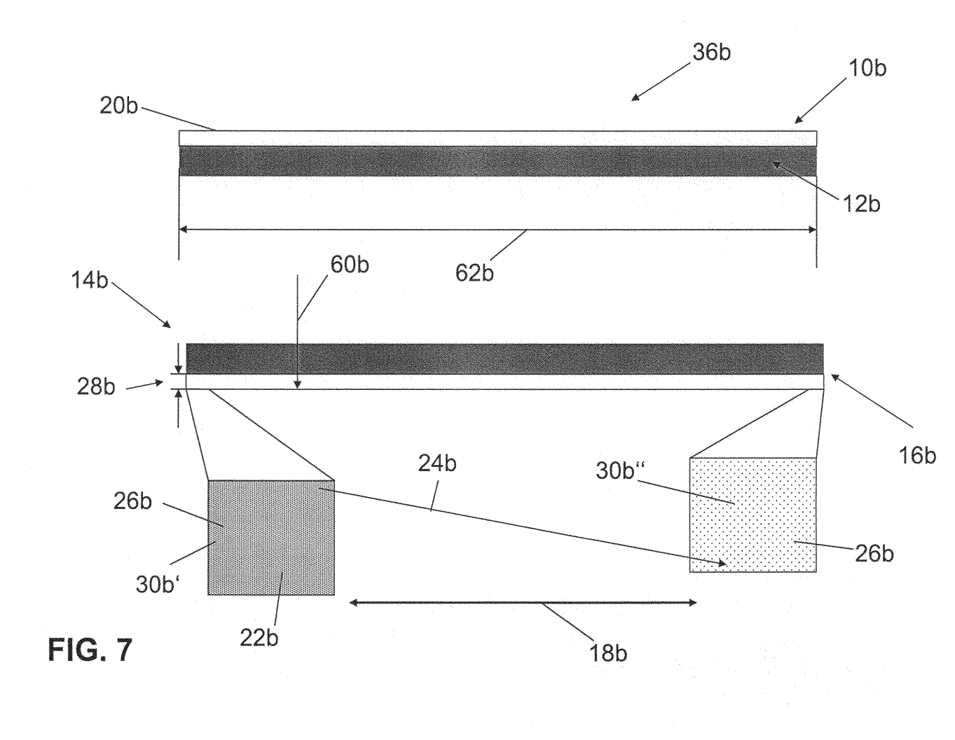

FIG. 7 shows an alternative exemplary embodiment of the medical implant 10a. Components, features and functions that remain the same are essentially labeled with the same reference numerals. To differentiate the exemplary embodiments, however, the letters a and b are added to the reference numerals of the exemplary embodiments. The following description is limited essentially to the differences in comparison with the exemplary embodiment in FIGS. 1 to 6, whereby reference can be made to the description of the exemplary embodiment in FIGS. 1 to 6 with regard to the same components, features and functions.

FIG. 7 shows an alternative medical implant 10b in the form of a stent 36b with a base body 12b of a chromium-cobalt steel. The base body 12b has a first end 14b and a second end 16b, which are arranged on opposite ends of the base body 12b in a main direction of extent 18b of the base body 12b. Furthermore, the base body 12b has a coating 20b, containing the immunosuppressant sirolimus with a narrow therapeutic window as the active substance 22b and a polymer matrix 56b of poly-L-lactide. Furthermore, the active substance 22b is introduced into the coating 20b with an active substance gradient 24b, so the quantity of active substance 26b in the main direction of extent 18b of the base body 12b decreases from the first end 14b to the second end 16b. This decline in the active substance gradient 24b is adjusted by means of several active substance concentrations 30b along the coating 20b. At the first end 14b, the coating 20b, which has a constant thickness 28b over the entire length 62b in the radical direction 60b, has a higher active substance concentration 30b′ than the active substance concentration 30b″ at the second end 16b.

The coating 20b may be applied by means of a method in which the polymer and the active substance 22b are mixed in different ratios prior to coating by means of a suitable mixing unit (not shown) such as a gradient pump, a multi-way valve, a conveyer screw. This mixing unit may also be provided as a dosing unit to regulate the concentration of the active substance.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. A medical implant comprising a base body which has a first end and a second end arranged opposite one another in a main direction of the extent of the base body, and a coating such that the coating has at least one active substance with an active substance gradient, characterized in that the quantity of active substance decreases from the first end to the second end at least in the main direction of extent of the base body.

2. The medical implant according to claim 1, characterized in that the active substance gradient varies with the thickness of the coating.

3. The medical implant according to claim 1, characterized in that the active substance gradient varies through changing active substance concentrations along the coating length from first to second end.

4. The medical implant according to claim 1, characterized in that at least one active substance is selected from a group consisting of:
lipid regulators (fibrates),
immunosuppressants,
immunomodulators,
vasodilators (sartans),
calcium channel blockers,
calcineurin inhibitors (tacrolimus),
antiphlogistics (glucocorticoids, cortisone, diclofenac),
anti-inflammatories (imidazoles),
anti-allergics,
oligonucleotides (dODN),
estrogens (genistein),
endothelializers (fibrin),
steroids,
proteins/peptides,
proliferation inhibitors,
analgesics,
antirheumatics,
cytostatics.

5. The medical implant according to claim 1, characterized in that the at least one active substance has a narrow therapeutic window.

6. The medical implant according to claim 1, characterized in that the first end is configured to be arranged in a cavity at an upstream end in an axial direction of flow in an implanted state.

7. The medical implant according to claim 1, characterized in that it is embodied as a stent.

8. The medical implant according to claim 1, characterized in that the base body comprises at least one of cobalt and chromium.

9. The medical implant according to claim 1, characterized in that the coating contains at least one polylactide.

10. The medical implant according to claim 1, characterized in that it is designed to elute at least one active substance in a human or animal body, at least one active substance being elutable, such that a desired local concentration distribution in an adjacent cavity wall is achieved.

11. The medical implant according to claim 1 made through a method comprising the steps of applying a coating stream with a polymer/active substance mixture at a variable velocity from the first end to the second end.

12. The medical implant according to claim 11, characterized in that the coating stream is moved at an increasing velocity from the first end to the second end.

13. The medical implant according to claim 11, characterized in that the coating stream is moved in at least one of the circumferential direction and in the axial direction relative to the medical implant.

14. A method for implanting the medical implant of claim 1 in an animal or human body, characterized in that the first end is arranged in a cavity in the animal or human body at an upstream end in an axial direction of flow.

15. A medical implant as defined by claim 1 wherein the active substance loading is at least 4 times greater at the first end than it is at the second end.

16. A medical implant as defined by claim 1 wherein the active substance loading varies linearly along the main direction of the implant.

17. A medical implant comprising:
a base body having a first end and an opposing distal second end in a main direction of the base body, the base body comprising at least one of cobalt and chromium; and,
a coating covering at least a portion of the base body and comprising at least one active substance, the coating active substance present in a quantitative gradient along the base body main direction and decreasing from the first end to the second end, the at least one active substance being elutable wherein a eluted active substance gradient concentration is achieved in an adjacent cavity wall when the implant is implanted in a cavity corresponding generally to the gradient concentration in the coating along the base body main direction.

18. A medical implant as defined by claim 17 wherein the at least one active substance is provided in a constant concentration in the coating and the coating thickness varies between the first and second ends to achieve the quantitative active substance gradient between first and second ends, and wherein the coating contains at least one polylactide.

19. An implant as defined by claim 17 wherein the coating is provided in a substantially constant thickness between the first and second ends and the concentration of the at least one active substance in the coating varies between the first and second ends to achieve the quantitative gradient.

20. An implant as defined by claim 17 wherein:
the at least one active substance has a narrow therapeutic window and comprises at least one of: lipid regulators (fibrates), immunosuppressants, immunomodulators, vasodilators (sartans), calcium channel blockers, calcineurin inhibitors (tacrolimus), antiphlogistics (glucocorticoids, cortisone, diclofenac), anti-inflammatories (imidazoles), anti-allergics, oligonucleotides (dODN), estrogens (genistein), endothelializers (fibrin), steroids, proteins/peptides, proliferation inhibitors, analgesics, antirheumatics, cytostatics; and,
the active substance loading at the first end is at least 4 times greater than the active substance loading at the second end.
